(19) United States
(12) Patent Application Publication
(10) Pub. No.: US 2017/0089887 A1
(43) Pub. Date: Mar. 30, 2017

(54) CONTRACTILITY ASSAY

(71) Applicant: Nano3D Biosciences, Inc, Houston, TX (US)

(72) Inventors: Glauco SOUZA, Houston, TX (US); Biana GODIN, Houston, TX (US); Monica LONGO, Houston, TX (US); Jerrie Selga REFUERZO, Houston, TX (US)

(73) Assignees: THE METHODIST HOSPITAL SYSTEM, Houston, TX (US); THE UNIVERSITY OF TEXAS HEALTH SCIENCE CENTER, San Antonio, TX (US)

(21) Appl. No.: 15/374,897

(22) Filed: Dec. 9, 2016

Related U.S. Application Data
(63) Continuation-in-part of application No. 13/995,610, filed on Jun. 19, 2013.
Application No. 13/995,610 is a continuation-in-part of application No. PCT/US2012/021233, filed on Jan. 13, 2012.
(60) Provisional application No. 62/265,884, filed on Dec. 10, 2015.
(60) Provisional application No. 61/438,310, filed on Feb. 1, 2011.

(51) Int. Cl. G01N 33/50; G06T 7/62; G06T 7/00; G01N 33/483; G06K 9/00

(52) U.S. Cl. CPC G01N 33/5061; G01N 33/4833; G06K 9/00134; G06T 7/0012; G06T 7/62; G06T 2207/10056; G06T 2207/30024

(57) ABSTRACT

Cells are magnetized and then grown in ring shaped 3D culture using a ring magnet. Contractility is measuring by tracking the size of the hole in the 3D cellular ring.

### PRIOR RELATED APPLICATIONS

This application claims priority to and is a CONTINUATION-IN-PART of 61/438,310, filed Feb. 1, 2011, WO2012106089, filed Jan. 13, 2012, and US20130280754, and also claims priority to 62/265,884, CONTRACTILITY ASSAY, filed Dec. 10, 2015, each incorporated by reference herein in its entirety.

### GOVERNMENT FUNDING

This invention was made with government support under Grant No: R41 HD081795 awarded by the NIH. The government has certain rights in the invention.

### FIELD OF THE DISCLOSURE

The invention relates to the field of contractility assays, drugs affecting contraction or relaxation, and materials and methods and specific applications relating thereto.

### BACKGROUND OF THE DISCLOSURE

Preterm birth, or delivery prior to 37 weeks, is a major health care issue that affects 11% of all pregnancies, or approximately 450,000 pregnancies in the United States. It is the leading cause of perinatal morbidity and mortality, accounting for 70% of neonatal deaths and 36% of infant deaths. Of the children that survive, 25-50% will suffer from long-term neurological impairment. These negative outcomes place an immense burden on families and healthcare payers in terms of the high costs of medical care, lost wages, and developmental disabilities associated with preterm labor.

Furthermore, preterm labor is not just a human problem, but also significantly affects agricultural production. Preterm labor difficulties in cattle, horses, sheep, goats, and pig farming operations all contribute to lost production and increased cost. As an example, drought stress, heat stress, high nitrate levels and other toxins in a cow's forage ration can trigger early calving, stillbirths and abortions. Indeed, some estimate the U.S. national costs associated with cattle stillbirths and abortion are estimated at approximately $64 million yearly for beef cow-calf producers and $27 million per year for dairy producers.

While we clearly understand the epidemiology of preterm labor and who would benefit most from improved intervention, the progression of preterm labor to preterm birth remains elusive. It is generally understood that increased uterine contractility is a precursor for preterm labor, but there is a lack of knowledge of the details of the physiology. As a result, the treatment of preterm labor has not advanced much in recent times, and preterm birth rates in humans have remained relatively constant over the past 30 years.

The typical treatment for preterm labor would be tocolytic therapy, whose function is to delay contraction long enough to administer corticosteroids to accelerate fetal lung maturation. Clinically available tocolytic therapies vary in drug class and mechanism and are currently prescribed empirically. Yet, their efficacy is based on genetic factors, and thus vary between different patients. Ideally, these therapies would be screened on a specific patient's tissue or cells to find the most effective tocolytic therapy for an individual gravid patient.

The common obstacle to understanding preterm labor physiology, discovering tocolytic therapies, and developing patient-specific therapeutic regimens is the lack of a model that both accurately and efficiently models uterine contractility. In vivo models are costly and time-consuming, and differences in how animals and humans give birth mean that pathological changes have different biological bases and responses to drugs. Moreover, recent pushes in government and industry, such as ICCVAM (Interagency Coordinating Committee on the Validation of Alternative Methods) and NICEA™ (NTP Interagency Center for the Evaluation of Alternative Testing Methods) by NIEHS, are forcing researchers to look for alternatives.

Ex vivo human and animal tissue models are accurate predictors of tocolytic efficacy, yet suffer from cost, regulatory hurdles, sample inconsistencies, and material scarcity. Additionally, these studies require extensive training and expensive equipment, which is sensitive to various factors and contributes to inaccurate data.

In vitro cell culture models are attractive alternatives as they are both easy and inexpensive. However, the majority of these models are two-dimensional (2D) cell monolayers that only poorly mimic native tissue environments. Their stiff plastic or glass substrates do not represent any tissue in the body, let alone uteri. They also mis-represent extracellular matrix (ECM) structure and composition, and the cell-cell and cell-ECM interactions that it supports. Lastly, biochemical and nutrient access is altered in 2D, as every cell has uniform exposure, unlike native tissues.

Potential solutions lie in three-dimensional (3D) cell culture platforms that can more faithfully recreate tissue structure and ECM composition in vitro. There are a variety of different platforms available for 3D-based methods, including: protein gels, such as Matrigel and collagen that recreate ECM composition; polymer scaffolds that reproduce tissue structure and material properties; hanging drop spheroids that use water tension in liquid droplets to aggregate cells into spheroids; round bottom plates that use plate geometry to aggregate cells in spherical bottom wells; and nanopatterned plates, with wells <1 μm patterned within the well where cells aggregate.

While 3D cell cultures using these systems can approximate native tissue, they can be expensive and time-consuming to fabricate, require specialized equipment or media, and be difficult to handle and retain samples. Additionally, these platforms are generally best in creating spheroids, but are not able to create different shapes that could reflect uterine contractility. Moreover, these platforms have difficulty in creating smaller 3D cell cultures in higher well number formats (384 and higher), which is critical when using a scarce resource, such as patient-derived cells. The ideal 3D cell culture platform for a precision assay to personalize preterm labor therapy would recreate a contractility model with speed, ease, and small cell numbers better than comparable 3D cell culture platforms and 2D models.

Thus, what are needed in the art are better 3D culture systems, methods and models for assessing contractility, and thus being able to predicting preterm labor and/or response to various drugs.

### SUMMARY OF THE INVENTION

To meet the unmet need for a robust, easy, yet physiologically reliable contractility assay, we have developed a new assay to mimic contractility in 3D rings using magnetic 3D bioprinting. The assay works by magnetizing cells and then using a ring magnet to print them into 3D rings using the magnetic field. The cellular ring thus formed will contract in a dose-dependent manner with simple and measurable metrics, and thus can be used to rapidly and easily screen tocolytic drugs. In preferred embodiments, the cells used are patient specific, thus providing an individualized prediction of a patient's response to various test reagents.

Such an assay could be used where ever contractility is an important parameter, such as in preterm labor, skeletal muscle contraction, smooth muscle contraction, cardiac muscle contraction, peristalsis, pre-eclampsia, eclampsia, other vascular disorders, fibroids, tumors, menstrual disorders, and the like. Further, although called a “contraction” assay herein, the same assay can of course be used to assess relaxation, which is a return of muscle fibers to their extended configuration.

The present invention thus provides an improved label-free, real-time cell contractility assay. The method uses 3D ring culturing by magnetic levitation and couples the 3D culture technique with image analysis to assess cell contractility and/or relaxation.

Generally speaking, the contractility detection method is based on the principal that 3D ring assemblies of cells will contract or relax in response to an agent, and that the contraction can easily be measured by measuring ring or hole size. Of course, once contracted, relaxation can also be measured. These measurements can be point measurements only, e.g., before and after stimulation, but can also be measured over time with e.g., video analysis or with the use of multiple images.

The method can be simply described, as follows:

1. The cells of interest are magnetized and levitated using a ring magnet to allow 3D culture growth. The ring magnet causes the cells to coalesce in a ring-like or toroidal shape, with a central hole.

2. The 3D rings are then exposed to a test agent.

3. Hole size is measured, either through still photos or via video footage, and this assessment is typically done before, during and after exposure, and can be done at a variety of exposure levels (agent concentration levels) and over time or at one or more particular time points.

4. The contractility versus agent level is plotted and thereby one can determine the effect of the test agent on contractility and/or relaxation.

Test agents can be anything whose contractile/relaxivity effect is desired to be assayed, including drugs, genetic materials (RNA, DNA, siRNA, miRNA), enzymes, proteins, cytokines, drug delivery systems, nanomaterials, particles, microparticles, nanoparticles, other cell types, cell components, suspected toxins or any environmental or industrial agent or chemical.

Although described in a linear, stepwise fashion above, this is only for ease of understanding, and usually all samples (zero dose agent control, plus multiple samples with increasing doses of one or more test agents) can be processed in parallel.

The assay can be completed very quickly, within 24 hours. In the alternative, samples can be grown for 1-7, preferably 2-3 days, to attain a sufficient ring size, and agent added at a later time point. Cells can be precultured (amplified) before ring printing, in either 2D or 3D, but preferably 3D. Cells can also be frozen before use. Combinations of the above are also possible.

Furthermore, the above method can be preceded by a cell collection step, thereby collecting cells from a particular gravid patient, or commercial sources of cells could be used. Patient cells can be collected by e.g., biopsy, by collecting material from prior labors, or surgeries, or as part of the evaluation for assisted reproductive technology (ART). Cells can be processed immediately or stored for up to 20 years prior testing.

Preferably, the photographs are taken while the cells are still levitating because this is expected to minimize disruption to the culture. However, this is not essential, and a 3D culture can be photographed when not levitating. This may be particularly appropriate for a contractility assay wherein one measures the rate of closure of a gap or hole (or the reverse) in a multicell structure.

In more detail, the invention is a contractility assay comprising culturing levitated cells to form 3D cellular ring structure having a hole therein, taking photomicrographs of said rings at one or more times, analyzing said photomicrographs to measure one or more of i) hole closure, ii) hole expansion, or iii) rates of change of hole size in either direction.

There can also be a plurality of samples of levitated cells, said samples being with and without one or more concentrations of various test agents or having test agents added at one or more times. Generally, a decrease closure rate with said test agent indicates that the test agent inhibits contraction, and wherein an increase closure rate indicates that said test agent stimulates contraction. Although we have exemplified the method using inhibition of contraction, obviously one could invert the assays, first contracting the cells (either by magnet removal or with the addition of a contractile agent) and then measuring an agent that allows relaxation.

The method can include culturing a plurality of 3D cultures for use as control cultures and a culturing a plurality of 3D cultures for use as test cultures, wherein a test agent is added to said test cultures and the effect of said test agent on said contractility or relaxation is then measured.

The method can also include adding varying amounts of said test agent to said a plurality of test cultures, taking a plurality of photomicrographs of said 3D cell culture at a plurality of times, and/or washing out said test agent and taking a further plurality of photomicrographs of said 3D cell culture at a further plurality of times.

In preferred embodiments, the cells are magnetized with a composition comprising: a) a negatively charged nanoparticle; b) a positively charged nanoparticle; and c) a support molecule, wherein one of said negatively charged nanoparticle or positively charged nanoparticle contains a magnetically responsive material, such as iron or iron oxide. The support molecule holds said negatively charged nanoparticle and said positively charged nanoparticle in an intimate disorganized or tangled admixture, e.g., not a micelle.

Preferably, the support molecule comprises peptides, polysaccharides, nucleic acids, polymers, poly-lysine, fibronectin, collagen, laminin, BSA, hyaluronan, glycosaminoglycan, anionic, non-sulfated glycosaminoglycan, gelatin, nucleic acid, extracellular matrix protein mixtures, antibody, or mixtures or derivatives thereof, b) wherein said negatively charged nanoparticle is a gold nanoparticle, and c) wherein said positively charged nanoparticle is an iron oxide nanoparticle. Most preferred, the composition is NANOSHUTTLE™ (Nano3D BioSci., Houston Tex.), e.g., poly-lysine, gold nanoparticles, and iron oxide nanoparticles.

The invention can include any one or more of the following embodiments, in any combination(s):

A contractility assay comprising:
a) obtaining contractile cells;
b) magnetizing said contractile cells;
c) magnetically creating a 3D ring of magnetized cells using a ring magnet, said 3D ring having
a hole;
d) taking photomicrographs of said 3D ring of magnetized cells at one or more times;
e) analyzing said photomicrographs to measure hole size or rate of change of hole size or both;
f) wherein hole size hole size or rate of change of hole size relates to contractility of said
contractile cells.
A contractility assay comprising:
a) obtaining contractile cells from a patient;
b) magnetizing said contractile cells;
c) magnetically creating a 3D ring of magnetized cells using a ring magnet, said 3D ring having
a hole;
d) adding a test agent to said 3D ring of magnetized cells;
e) measuring a rate of change of hole size, wherein rate of change of hole size relates to
contractility of said contractile cells.
A uterine contractility assay comprising:
a) obtaining contractile cells from a uterus;
b) magnetizing said contractile cells;
c) magnetically creating a 3D ring of magnetized cells using a ring magnet, said 3D ring having
a hole;
d) adding a test agent to said 3D ring of magnetized cells;
e) taking photomicrographs of said 3D ring of magnetized cells at one or more times before and
after adding said test agent;
f) analyzing said photomicrographs to measure rate of change of hole size;
g) wherein rate of change of hole size relates to contractility of said contractile cells.
A uterine contractility assay comprising:
a) obtaining contractile cells from a uterus;
b) magnetizing said contractile cells;
c) magnetically creating a 3D ring of magnetized cells using a ring magnet, said 3D ring having
a hole;
d) adding a test agent to said 3D ring of magnetized cells;
e) measuring rate of change of hole size;
wherein rate of change of hole size relates to contractility of said contractile cells.
Any method as herein described, further including culturing a plurality of 3D rings for use as
control cultures and a culturing a plurality of 3D rings for use as test cultures, wherein a test agent
is added to each of said test cultures.
Any method as herein described, further including adding varying amounts of said test agent to
said plurality of test cultures.
Any method as herein described, comprising taking a plurality of photomicrographs of said 3D cell
culture at a plurality of times.
Any method as herein described, further comprising washing out said test agent and taking a
further plurality of photomicrographs of said 3D cell culture at a further plurality of times.
Any method as herein described, wherein cells are magnetized with a composition comprising: a)
a negatively charged nanoparticle; b) a positively charged nanoparticle; and c) a support
molecule, wherein one of said negatively charged nanoparticle or positively charged nanoparticle
contains a magnetically responsive element, and wherein said support molecule holds said
negatively charged nanoparticle and said positively charged nanoparticle in an intimate and
disordered admixture (not a micelle).
Any method as herein described, wherein the support molecule comprises peptides,
polysaccharides, nucleic acids, polymers, poly-lysine, fibronectin, collagen, laminin, BSA,
hyaluronan, glycosaminoglycan, anionic, non-sulfated glycosaminoglycan, gelatin, nucleic acid,
extracellular matrix protein mixtures, antibody, or mixtures or derivatives thereof, wherein said
negatively charged nanoparticle is a gold nanoparticle, and wherein said positively charged
nanoparticle is an iron oxide nanoparticle.
Any method as herein described, wherein the composition comprises poly-lysine, gold
nanoparticles, and iron oxide nanoparticles.
Any method as herein described, wherein said contractile cells are obtained from a patient.
Any method as herein described, wherein said contractile cells are obtained from a patient's
uterus, preferably a human patient.
Any method as herein described, further including culturing a plurality of 3D rings for use as
control cultures and a culturing a plurality of 3D rings for use as test cultures, wherein a test agent
is added to each of said test cultures.
Any method as herein described, wherein contraction is initiated by removing said ring magnet.
Any method as herein described, wherein contraction is initiated by adding a contractile agent.
An assay device comprising a microtiter plate having a plurality of wells, each well containing a
culture medium containing a 3D ring of magnetic contractile cells. Each said 3D ring of magnetic
contractile cells can be floating in said culture medium, or can be printed onto a bottom of a well.
Any assay device as described herein, said contractile cells being uterine derived mesenchymal
cells or smooth muscle cells, preferably obtained from a uterus of a patient.
Any method as herein described, wherein said contractile cells or smooth muscle cells are from a
heart, uterus, lung, stomach, intestine, urogenital tract, placenta, maternal vasculature, umbilical
cord etc.
Preferably, there are about 50,000-200,000 cells/ring, or about 100,000 cells/ring, although there
is a strong desire to reduce cell number if patient cells are scarce, and 10,000 or 25,000 cells
may suffice, or even 1-5000 with sufficient magnification.
A uterine contractility assay comprising:
a) obtaining contractile cells from a myometrium of a patient's uterus;
b) allocating said contractile cells to a plurality of wells in a microtiter plate;
c) magnetizing said contractile cells;
d) magnetically creating a 3D ring of magnetized cells using a ring magnet, said 3D ring
having a hole, said hole having a size;
e) adding a test agent to said 3D ring of magnetized cells;
f) initiating contraction by removing said ring magnet; either before or after step e);
g) taking photographs of an entirety of said microtiter plate at one or more times before and
after adding said test agent;
h) analyzing said photographs to measure a rate of hole size contraction;
i) wherein a reduced rate of hole size contraction means said test agent inhibits
contraction.
Any method as herein described, wherein said microtiter plate is a 384-well microtiter plate, and
wherein 10-100 × 10³ or 75 × 10³ cells/well are used to form said 3D ring.
Any method as herein described, wherein said cells are grown in 3D culture for 1-7 days,
preferably 2-3 days, to form a 3D spheroid, wherein said spheroid is broken up to provide
contractile cells to be used for 3D ring formation.
Any method as herein described, wherein said cells are obtained from a patient, and frozen for
later use.

“Cultures” and “samples” are used interchangeably herein since the samples comprise live cells, usually in a culture medium. However, any suitable liquid that maintains cell structure and viability could be used, and we do not mean to imply by the word “culture” or “culturing” that exponential growth is a requirement.

As used herein, a “magnetic driver” is a lid or cover that can fit over or under a culture plate and has magnets permanently or reversible affixed thereto, such that magnetic driver can be used with the plate to levitate magnetic cells being cultured in the plate.

As used herein, a “contractile cell” is a cell that will contract under stimulation when grown in a 3D culture. Cells from the uterus (uterine myocytes) and heart (cardiomyocytes) are examples thereof. Skeletal muscle cells, cardiac muscle cells, and smooth muscle cells are also examples.

By “printing” or “bioprinting” herein, we mean using cells as a living “ink” to print patterns, such as cell rings on a vessel surface, or rings in a culture medium, as is done herein.

By “culturing” herein, we include culturing single cell types or co-culturing more than one cell type.

By “patient” herein, we mean any mammalian patient, but preferably referring to humans.

By “contractile” assay herein, we also include the inverse—a relaxation assay (e.g., negative contractility).

By “magnetically printing” a 3D ring, we mean applying a magnetic field in a ring shape to a collection of cells, such that the cells coalesce and form a 3D ring or toroidal structure. Typically, this is done by putting magnets under the cells, but the magnet can be over or under according to convenience.

When used herein, a “3D ring” of cells refers to a roughly annular or toroidal ring of cells having a hole in the center. Such cells are convenient for measuring contraction, but it is possible that a linear arrangement (cylinder or rod) of cells or other shapes could serve the same function. The 3D ring of cells can be assayed herein while levitating or while resting on the bottom of a cell plate, as preferred.

As used herein “vessel” or “well” refers to any container for culturing cells, such as a Petri dish, flask, or multiwell culturing plate.

By “over said well” we mean that the magnet cannot dip into the culture media when the magnet is in use, but sits over the culture media, not contacting same. When the driver is under the plate, the media is likewise not contacted.

By “microplate” or “microtiter plate” or “multiwell” plate or vessel what is meant is the industry standard microplate. Note that ANSI-SLAS publishes standard sizes for microtiter plates in order to ensure interoperability to robotics and multi-pipettors, and these can be found at slas.org/resources/information/industry-standards/.

By “myometrial cells” or “myometrial smooth muscle cells” what is meant are cells that originate from the myometrium. The myometrium is the middle layer of the uterine wall, consisting mainly of uterine smooth muscle cells (also called uterine myocytes), but also of supporting stromal and vascular tissue. Its main function is to induce uterine contractions.

Although we currently used standard microtiter plates for the assay, technology continues to evolve, and this technology will soon be outdated as disposable lab-on-chip devices with embedded microfluidics become more and more common. Plates could also be designed with electromagnetic drivers, thus eliminating the bulk and expense of rare earth magnets. Smart plates may also be common in the future, eliminating the need for photos, as capacitive or impedence sensors automatically determine ring size. See e.g., U.S. Pat. No. 7,459,918, incorporated by reference herein in its entirety for all purposes.

“Magnet” refers to any material creating a magnetic field and can be a permanent magnet or an electromagnet.

Preferably, a rare earth magnet is employed. Examples of rare earth magnets suitable for use with the present invention include, but are not limited to, neodymium rare earth magnets, samarium-cobalt rare earth magnets, Nd₂Fe₁₄B, SmCo₅, Sm(Co,Fe,Cu,Zr)₇, YCO₅, or any combination thereof.

Neodymium rare earth magnets are the strongest and most affordable type of permanent magnet, and are generally preferred, but samarium-cobalt magnets have a higher Curie temperature (the temperature at which the material loses its magnetism) and may be preferred for uses involving high sterilization temperatures.

Particular types of rare earth magnets may also be selected as desired according to the conditions to which the rare earth magnets may be exposed. For example, any of the following factors may be considered in selecting a type of rare earth magnet: remanence (Br) (which measures the strength of the magnetic field), coercivity (Hci) (the material's resistance to becoming demagnetized), energy product (BHmax) (the density of magnetic energy), and the Curie temperature (Tc). Generally, rare earth magnets have higher remanence, much higher coercivity and energy product than other types of magnets. Where high magnetic anisotropy is desired, YCO₅ may be suitable for use.

In place of or in addition to the rare earth magnets, powered magnets may be incorporated into the devices of the invention, and batteries may be used to power the powered magnets as desired. Alternatively, RF or other electromagnetic radiation activated power sources can be used to power the magnet, such as is used with RFID tags. However, for simplicity, durability, and cost reasons, the permanent magnet is preferred. It is possible, however, that with decreasing well size, it may eventually be preferred to use electromagnetic means for levitating magnetic cells.

We have tested a number of permanent magnets, both in modeling studies and in real experiments, and can elaborate a number of principals for the selection of magnetic size, strength and shape. Firstly, the magnet size is confined by the size of the plates with which it will be used, as excess magnet is a waste of resources.

Second, the distance of the magnet from the cells can vary with increasing field strength, stronger magnets being held farther away than weak magnets, and generally the magnets being positioned so as to not touch the media or an intervening cover (if used).

These considerations must be balanced against the lifting height of the magnet (how far away the magnet can be and still lift cells), as well as the desired growing height. Magnetic field interference between adjacent magnets is also important in designing multi-magnet holders, and it is preferred that the magnets of multiwall plates be positioned so as to alternate polarity and thus minimize interference. Additionally, meniscus effects from the media surface shape become increasingly important in plates with increasing well number.

Third, our results indicate that the gradient and field strength produced by each permanent magnet are important considerations, and that a steep gradient and high field strength serve to minimize interference between magnets and still provide good lifting and growing heights. See e.g., U.S. Serial No. US20160137974, MICROPLATES FOR MAGNETIC 3D CULTURE, filed Dec. 10, 2015, incorporated by reference herein in its entirety for all purposes.

In general, a fairly strong ring magnet is needed, providing about 12000-15000 Br_{Max} at a distance of 1-2 cm from the culture. We tested candidate magnets for a 35 mm plate that included various disc magnets from K&J MAGNETICS®. MM-A-32 is an annular (ring) shaped magnet of 1.26″ (32 mm)×0.32″ (8 mm) with a small tapered countersunk central hole 0.22-0.39″. It is a Grade 38 NdFeB magnet with Ni—Cu—Ni coating, axial poles, a pull force of 55.1 lbs, Brₘₐₓ of 12,600 Gauss and BHₘₐₓ of 38 MGOe. MM-A-20 is very similar, 0.79″×0.28″, hole 0.18-0.33″, but due to its smaller size having a pull force of only 13.20 lbs, Brₘₐₓ of 12,600 Gauss, and BHₘₐₓ of 38 MGOe. These magnets can be used for a standard six well plate (127.76 mm×85.47 mm, wells are 35.43 mm×17.4 mm). Additional magnets that can be used in microtiter plates include the following:

| | | | | | Pull | Surface | | | |
|---|---|---|---|---|---|---|---|---|---|
| | | Wt | | Magnetization | Force | Field | Brₘₐₓ | Bhₘₐₓ | |
| Name | Details (inches) | (oz) | Material | direction | (lbs) | (Gauss) | (Gauss) | (MGOe) | Grade |
| 96 well | 0.1875 × 0.0625 × 0.1875 | 0.02 | NdFeB | Axial | 1.85 | N/A | 13200 | 42 | N42 |
| 384 well | 0.125 × 0.0625 × 0.25 | 0.01 | NdFeB | Axial | 0.84 | N/A | 14800 | 52 | N52 |

The following abbreviations may be used herein.

| Abbreviation | Definition |
|---|---|
| ART | Assisted reproductive technology |
| AUC | Area under the curve |
| BrEpic | Bronchial Epithelial |
| ECM | Extracellular matrix |
| FBS | Fetal Bovine Serum |
| HEK | Human Embryonic Kidney |
| HPF | human primary fibroblast |
| HUtSMC | Human Uterine Smooth Muscle Cells (HUtSMC) |
| MLM | magnetic levitation method |
| MTT | 3-(4,5-dimethylthiazol-2-yl)-2,5-diphenyl tetrazolium |
| | bromide], used in a prior art cell viability or proliferation |
| | assays based on enzymatic conversion of the substrate |
| | MTT to a purple color. |
| NIR | Near-infrared spectroscopy |
| PR | Preference ratio |
| SMC | smooth muscle cells |
| SMCM | smooth muscle cell medium |

### BRIEF DESCRIPTION OF THE DRAWINGS

FIG. 1A-F. Photomicrographs of human primary fibroblast (1A and 1D; HPF; 500,000 cells) and smooth muscle cells (1C and 1F; SMC; 500,000 cells) cultures and co-culture (1:1; HPF-SMC) of human primary fibroblast (250,000 cells) with smooth muscle (250,000 cells) cells (1B and 1E) grown in 3D showing hole closure as a function of time (3, 12, 20, and 34 hours) when cultured in smooth muscle (1A-C, left) or fibroblast (1D-F, right) media (SCIENCELL RESEARCH LAB.,™ Carlsbad, Calif.; Fibroblast Medium, Catalog Number: #2301; and Smooth Muscle Cell medium, Catalog Number: #1101 media; 2.5× magnification, scale bar is 300 μm).

FIG. 2A. Schematic of magnetic 3D ring bioprinting.

FIG. 2B. Uterine rings assembled using Nano3D technology. Myometrial SMC cell rings using SMC-A (SMC from patient A) printed at varying cell concentrations as captured by the iPod Touch (left), and their area measured by the Python-based analytical software (right). Full rings were detectable by the software starting at 100,000 cells/ring, which was used as the cell concentration for this assay. Scale bar=5 mm.

FIG. 2C. The contraction of uterine rings. SMC-A ring area as measured in pixels as a function of time at various cell concentrations. Note that the rings contracted immediately after printing, suggesting that the levitation and printing times of 2 and 1 h, respectively, were sufficient to produce a contractile ring. Cell/ring values in legend are in thousands.

FIG. 3. n3Dock with iPod. In this compact apparatus, an iPod is used to image a whole plate of spheroids above it at pre-programmed intervals for the length of the experiment. This system avoids the need to image and measure each well individually under a microscope, improving throughput and efficiency.

FIG. 4A-C: Schematic of the contractile assay, called “C—BiO Assay” herein. FIG. 4A. Contraction can be imaged with the iPod using a commercial device (“n3Dock”) specially designed to fold a microtiter plate at the correct distance from a camera source, such as an i-phone (described in WO2013163059 and US20150091233, incorporated by reference herein in their entirety for all purposes). Exemplary images are shown in FIG. 4B. By assessing hole size, one can detect tocolytic effects of compounds, such as nifedipine, on myometrial SMC contraction. The C—BiO Assay was demonstrated with nifedipine and the results are shown in FIG. 4C.

FIG. 5: Harvest efficiency for myometrial SMCs using different methods of freezing. The cryobox method was the closest to matching the efficiency from fresh tissue.

FIG. 6: Time series of patient-derived myometrial SMCs exposed to various tocolytic drugs. Different cell types had different responses to the drugs. When thawed from frozen tissue rather than freshly digested tissue, similar responses were seen to tocolytics.

FIG. 7: Direct comparison of contraction inhibition response as measured by the CBiO Assay and wire myography. Contraction inhibition response as measured by the CBiO Assay (hatch) and wire myography in response to indomethacin (dark) and nifedipine (light).

### DETAILED DESCRIPTION

In prior work, Nano3D Biosciences developed materials for magnetizing cells, levitating cells and growing 3D cultures, as well as tools to handle and manipulation such cells and cultures and applications for same.

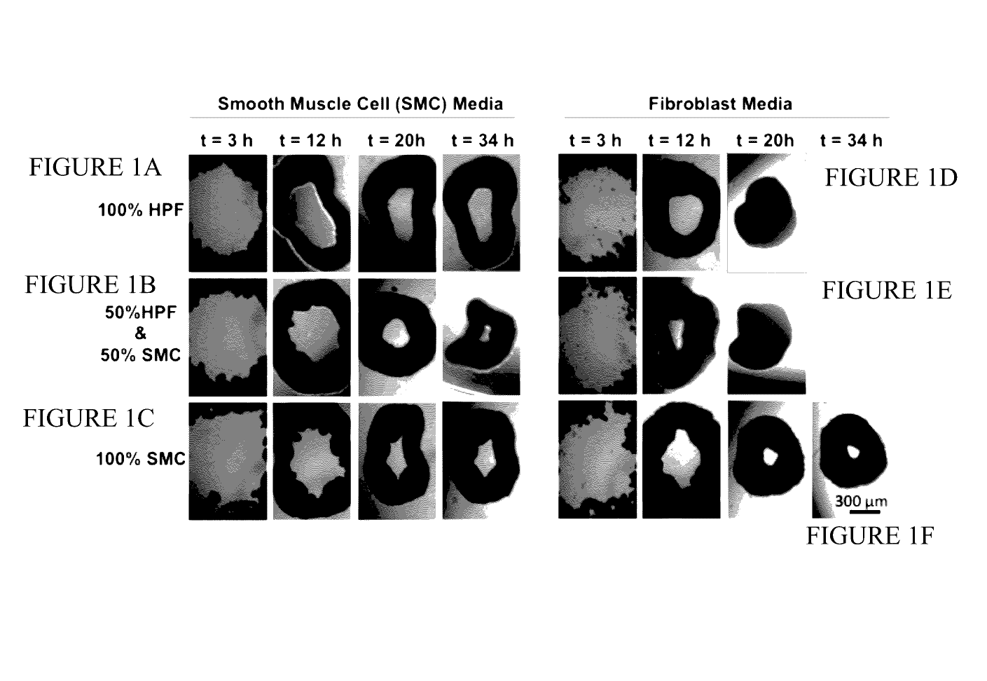

FIG. 1A-F, for example, shows photomicrographs of a wound healing or wound healing assay using HPF, SMC, and co-culture HPF of SMC (HPF-SMC) cells. Two different media conditions were tested, HPF and SMC media, for wound closure generated with the magnetic patterning approach described in US20130280754, and the holes were monitored over time. As a function of time, cell migration, 3D culture contraction, and new cell growth caused the holes to close up.

Wound sizes (aka hole size) decreased with both media types within the period of 34 hours, but wounds of the cultures in SMC medium (FIG. 1A-C) did not completely close over the period of 34 hours. Next, SMC cultures (FIGS. 1C and 1F) were not sensitive to media type since these cultures under both media types did not close completely over the 34 hour period. When comparing wound closure of HPF (FIGS. 1A and 1D) cultures and HPF-SMC co-cultures (FIGS. 1B and 1E), the wounds of the cultures in fibroblast media (FIG. 1D-E) closed completely under 20 hours in contrast to cultures in SMC media, which did not close completely over 34 hour period. These results suggest that HPF cells are sensitive to the two different media types and wound closure is accelerated, shown by complete closure of the ring by 20 hours when cells are cultured in HPF media.

We have now realized that the above assay provides a basis for the development of a contractility assay, with applications in assessing any contraction or relaxation of tissue, and with a particularly useful application in assessing the effect of test agents on uterine contraction and thus preterm labor.

Since the contractility assay is only interested in contraction, not growth, the assay is much faster, performed in 1-5 hours, as opposed to the days of the wound closure assay.

We envision that samples from routine procedures taken from labor such as placenta and biopsies of the uterus will be taken to produce an assay that: (1) is fast, reliable, and representative of the structure and physiology of the mammalian myometrium; (2) predicts clinically observed tocolytic and relaxant effects on the myometrium/maternal endothelium in vitro; (3) requires a significantly shorter timeframe; (4) does not require any additional equipment; and, (5) enables reproducible and accurate high-throughput analysis compared to other methods.

To achieve this goal, we designed an assay based on a recently explored methodology for 3D cell culture and magnetic 3D bioprinting, to create structures resembling tissue microenvironments. The advantage of magnetic 3D bioprinting to generate 3D cultures lies in the fact that 3D cultures can be rapidly printed within hours using magnetic forces and without the need of any special equipment, media, or growth factors, making it a cost-effective technique that can be used with routine supplies found in any cell culture laboratory.

3D bioprinting has now been used to magnetically print cells into a 3D ring that differentially contracts in response to differing agents. Contraction is measured using a mobile device-based imaging system that is programmed to image multiwell plates of rings at specific time-points, forgoing the need to painstakingly image each individual well under a microscope and the need for large, expensive real-time imagers. Additionally, contraction is label-free, so it does not require any specialized reagent or equipment, and the rings can also be experimented post-assay using immunofluorescence or gene expression profiling for high-content testing.

This assay holds the potential not only to efficiently screen tocolytic therapies, but also as an assay to personalize tocolytic therapies for specific patients, which is significant given the high risk of recurrence for preterm labor.

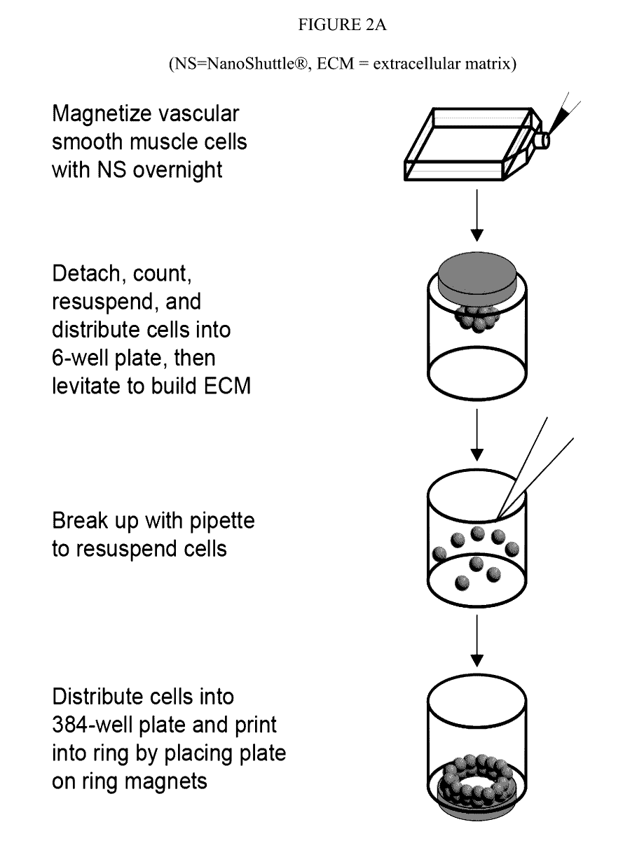

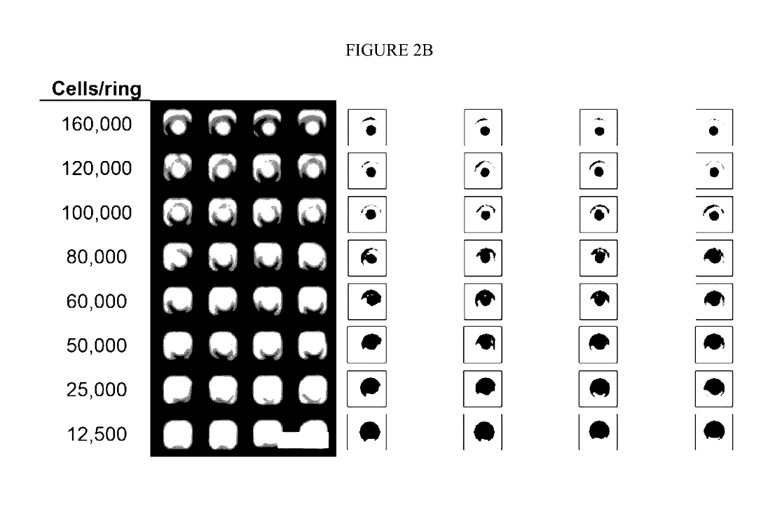

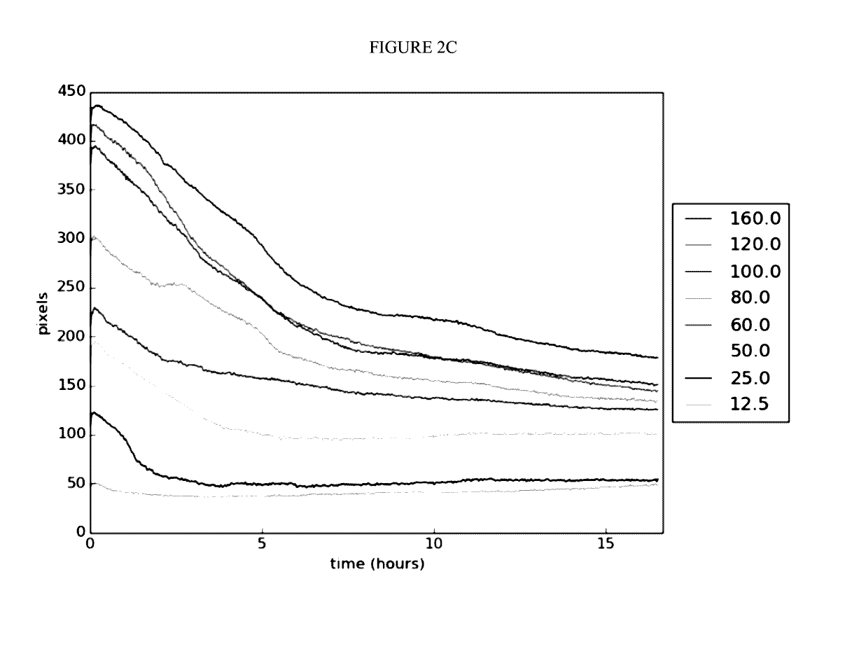

Our 3D contractility assay works by magnetizing, and then printing cells using magnetic field in a ring shape. The magnetic forces aggregate the cells, where without a stiff substrate to attach to, the cells interact with each other to form a larger 3D structure with culture-generated ECM (FIG. 2A).

Cells, preferably patient derived cells, are magnetized by their incubation with NanoShuttle™ (“NS” Nano3D Biosciences, TX), a commercially available magnetic nanoparticle assembly (˜50 nm) consisting of gold, iron oxide, and poly-L-lysine (PLL), in the form of tangled, matt-like or felt-like intimate admixture of the components. Via PLL, the NS electrostatically and nonspecifically attaches to cell membranes (˜50 pg/cell) to magnetize cells, and remains attached for about 8 days, after which the NS will release off the cell into the ECM.

NanoShuttle™ is biocompatible—there has been no deleterious effect by NS on proliferation, viability, and inflammatory and oxidative stress detected to date. Moreover, NS has not been shown to interfere with any fluorescent or luminescent endpoint, nor affect nor hinder genetic analysis. The magnetic field strengths and forces (1 to 60 pN/cell, preferably 10-50, 20-40 or about 30 pN/cell, and 1 to 150 G, preferably 50-125, or about 100 G) used to print magnetized cells have also been shown to have no effect on proliferation, viability, inflammatory and oxidative stress.

The advantages of using magnetic 3D bioprinting for developing a uterine contractility assay compared to other 3D cell culture platforms are numerous. Biologically, aggregating cells to form toroids uses the cells' intrinsic ability to generate ECM that is relevant to itself, unlike artificial protein gels like Matrigel or collagen whose foreign composition could potentially skew results and is subject to inconsistencies in composition. This is crucial given the wealth of knowledge in biomedical research on how ECM can influence cell behavior, particularly with regards to reproductive tissue and preterm labor.

On the technical side, magnetic 3D bioprinting is rapid and easy to use. The use of magnetic forces helps to accelerate 3D cell culture formation, from 1-3 days in other platforms to 24 hours maximum, and even as fast as 1-2 hours. Magnetic forces have the added ability to hold down and transfer 3D cultures, which is critical given that in other platforms, where spheroids are unattached or loosely attached, and routine cell culture maintenance and processing for staining is difficult without disrupting or losing the 3D culture.

Additionally, since the magnets and their fields are fixed with respect to the culture plate, 3D cultures can be reproducibly created or printed based on the shape of the magnet. This attribute is critical for this proposal, as other 3D platforms are unable to create or print consistent rings that mimic uterine contractility.

Lastly, as magnetization occurs on the individual cell level, the size of these rings can be adjusted by cell number and magnet shape to fit higher throughput formats like 384-well plates. Overall, our magnetic 3D system is the ideal cell culture platform to develop a uterine contractility assay for preterm labor management.

We developed an assay, the CBiO Assay (contractility BiO Assay) to mimic uterine contractility in vitro. The basis of this assay is one in which cells are magnetically 3D bioprinted into 3D rings (e.g., 0.125″ outer diameter, 0.0625″ inner diameter) in 384-well plates. After printing, the magnetic field is removed, and these rings contract immediately (<24 h), as cells within the ring contract, rearrange, and compact the ring.

Ring contraction on its own, without any test agent, will typically be the negative control. The rate or level of contraction for cultures with test agent will be subtracted from and/or divided by the contraction of the negative control, where the ratio of test agent/negative control >1 and/or the subtraction of test agent—neg. control <0 means the test agent induces contraction. On the other hand, when the ratio of test agent/negative control <1 and/or the subtraction of test agent—negative control >0, the test agent slows contraction. Next, if test agent induces dilation, rings increase in size over time, the ratio of test agent/negative control >1 and/or the subtraction of test agent—negative control >0 means the test agent induces dilation. However, there are other ways of setting up the assay, and this is exemplary only.

When we allowed the 3D cell rings to contract, we found that they contracted immediately after printing (FIG. 2C). The fact that they contracted immediately suggests that cells within the ring rearranged into a contractile state during levitation and printing so that the ring could be contracted once the magnet was removed. These results show that the 2 hr of levitation time and the 1 hr of printing time were sufficient to bioprint contractile rings, and these parameters will be used for our prototype work herein. However, variations in time are possible (e.g., 1-48 hr for each step, preferably 1-24, 1-12, 1-6 or so hours).

Of course, the cells can be amplified to a suitable number before the assay is initiated, e.g., by 2D culturing or 3D culturing for some period of time (1-7 or 1-3 days). Preferably, the cells are magnetically 3D cultured to form spheroids with relevant ECM, and the spheroids disaggregated for 3D printing of rings.

The endpoint—e.g., rate of contraction or change in size of ring at a single time point—can be easily measured by recording the size of the ring over time. This label-free endpoint escapes the limitation of other reagent-based assays that suffer from poor reagent diffusion and light penetration through dense 3D cultures that underreport biological events to capture a culture-wide drug response. Moreover, the rate of contraction was found to be dose-dependent, as demonstrated with the wound healing of hepatocytes (Timm, 2013). In our prototyping, we developed a version of this assay as the CBiO Assay using primary human myometrial smooth muscle cells.

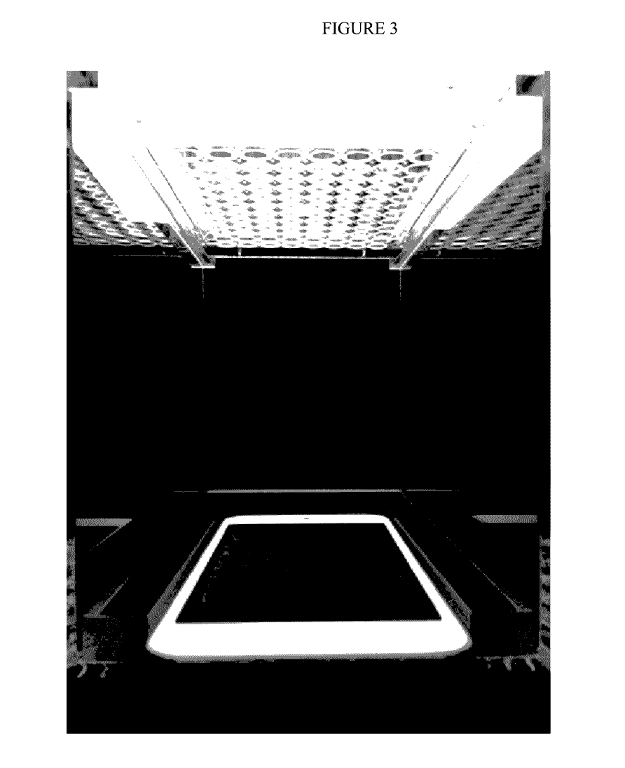

Along with this CBiO assay, a mobile device-based imaging system (n3Dock) was used to image and measure whole plates of rings (FIG. 3). This imaging system is driven by an iPod Touch (Apple Computer, CA) that was programmed to automatically image plates at regular intervals (≧1 s) for the length of the experiment using a freely available app for download (Experimental Assistant, Nano3D Biosciences). Using the n3Dock to image rings is possible given the fine camera resolution of the iPod (5 MP, resolution=250 μm) (Timm, 2013), and the brown color of the ring imparted by the NS that provides contrast against the lightness of the surrounding media. As a result, the iPod captures images of whole plates in a compact dock that fits within most standard incubators. Of course, any smart phone or pad or tablet equipped with a camera could be used herein.

In imaging whole plates, the n3Dock avoids having to image and measure each ring individually under a microscope or with an expensive real-time imaging instrument. The images can then be moved wirelessly to a computer for batch analysis with Java-based software (Tseng, 2015). All told, this CBiO Assay can print, assay, image, and analyze 3D myometrial smooth muscle rings in less than 24 hours.

Where the magnet is left in place for the assay, we usually place a white piece of paper on top of the magnet to hide the dark background of the magnet, and use an under-plate magnet. With the magnet in place, contraction rates are usually reduced, probably due to the magnetic field counteracting the contractile forces, but contraction is still measurable. However, typically, we remove the magnet to initiate contraction, thus being able to record an amplified effect. In those cases where cells contract too quickly, the magnet can be left in place to slow the effect down. In either case, a white background helps with imaging, at least for these cell types. A lit background may also prove beneficial, in which case a lit screen can be placed under the plate, or specialized equipment could be provided instead.

The images were transferred, either by wired or wireless connection, to a computer where analytical software that developed in Python was used to analyze all images at a rate of 60,000 data points/min. While originally designed around an iPod, the fundamental aspects of this system can be transferred to any mobile device and operating systems, including Android and Windows-based phones.

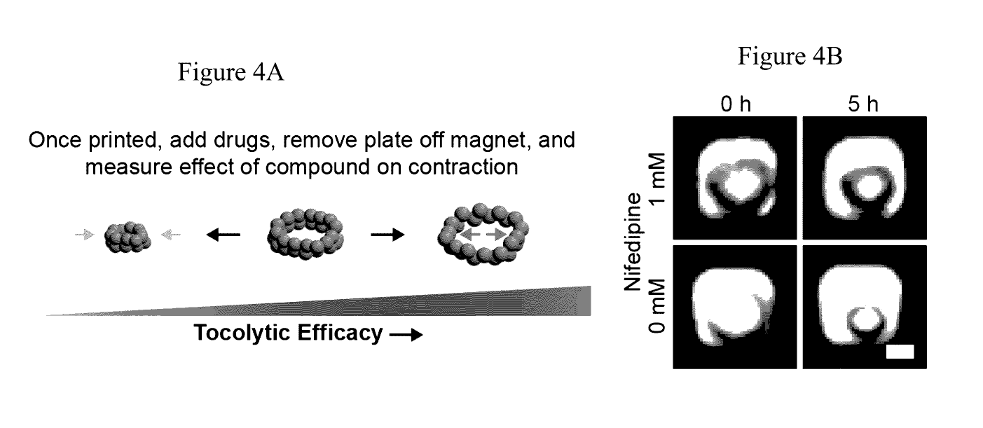

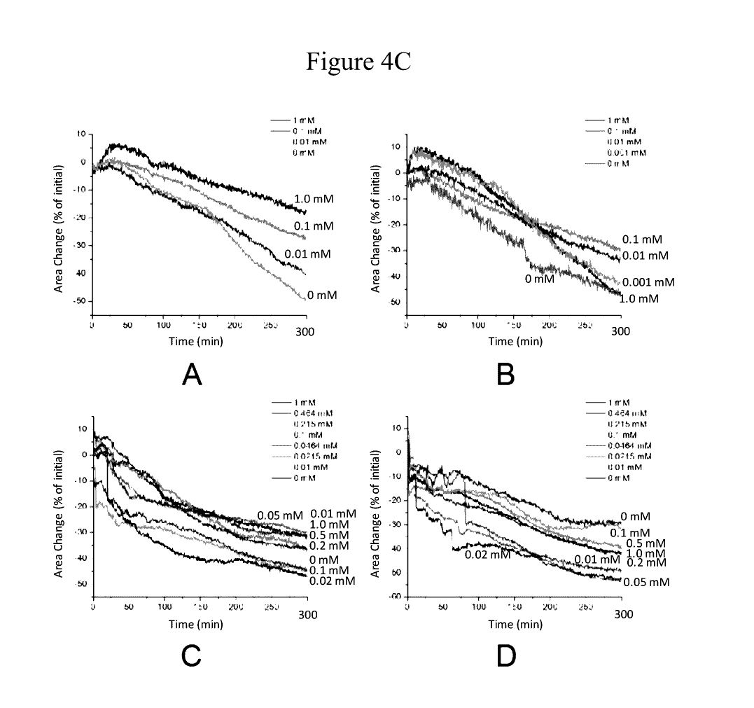

In our initial work, we developed and validated a prototype CBiO Assay (FIG. 4). First, we optimized the printing of myometrial smooth muscle cells into rings in 384-well plates. In particular, we found the cell number and printing time that yielded a contractile ring that contracted immediately after printing.

Next, we assayed the myometrial smooth muscle cells with several tocolytic drugs, and we were able to detect a tocolytic effect by observing slowed ring contraction with exposure to higher concentrations of the test compound. Moreover, we used commercially available human myometrial smooth muscle cells (PromoCell, Germany, catalog. no. C-12576) sourced from different patients, and found variance in the response as a function of cell source. These results with the CBiO Assay demonstrate our ability to detect variations in tocolytic efficacy on different patients' cells.

In the next phase of work, we developed methods for the cryopreservation and storage of patient-derived uterine tissue. Cryopreservation of uterine tissue would be most beneficial to mothers who undergo preterm labor, and are thus at higher risk of preterm labor in their next pregnancy. Their myometrial smooth muscle cells could be excised and banked, to be assayed later and determine their tocolytic regimen in time for their next pregnancy. Moreover, cryopreservation would avoid an immediate rush after extraction to ship the tissue and either assay with ex vivo testing, or digest into cells for in vitro cell culture. The main parameters to be studied are the type of cryoprotectant and the method of cryopreservation.

Uterine tissue was obtained from consenting women undergoing scheduled cesarean sections at term gestation greater 37 weeks. These biopsies (2×2×4 cm) were immediately placed in Hank's balanced salt solution (HBSS). The tissues were then cut into four pieces and weighed. Three of the pieces were then cryopreserved in three separate ways: flash freezing, in which the tissue was transferred immediately to a liquid nitrogen tank for long-term storage; slow freezing, where the tissue was frozen stepwise at 4° C. for 20 min, 80° C. overnight, and then in liquid nitrogen; and the cryobox method, in which the tissue was placed immediately into a CoolCell (Biocision, CA) to freeze overnight at −80° C., then transferred to liquid nitrogen. In all three cases, the cryoprotectant was 10% DMSO in SMC medium. The remaining piece of tissue not frozen was immediately harvested for cells as control.

After 1 month of storage, the tissues were thawed and the cryopreservation medium was replaced with HBSS without calcium and magnesium, and finely minced. The tissues were then digested into cells with the addition of 0.25% trypsin and 0.5% DNAse (final concentrations—0.1% trypsin, 0.1% DNAse in HBSS) for 30 min incubation in a shaking incubator at 37° C. Next, the tissues were centrifuged (400 g, 5 min) with the supernatant replaced with HBSS containing 0.2% collagenase. The tissues were digested for another 30 min at 37° C. Once digested, the cells were filtered out of the tissue and centrifuged (400 g, 5 min), then washed in RPMI 1640 with 10% fetal bovine serum (FBS) and 1% penicillin/streptomycin. Finally, the cells were plated onto a T75 flask and cultured for 7 days with daily media changes for the first 3 days.

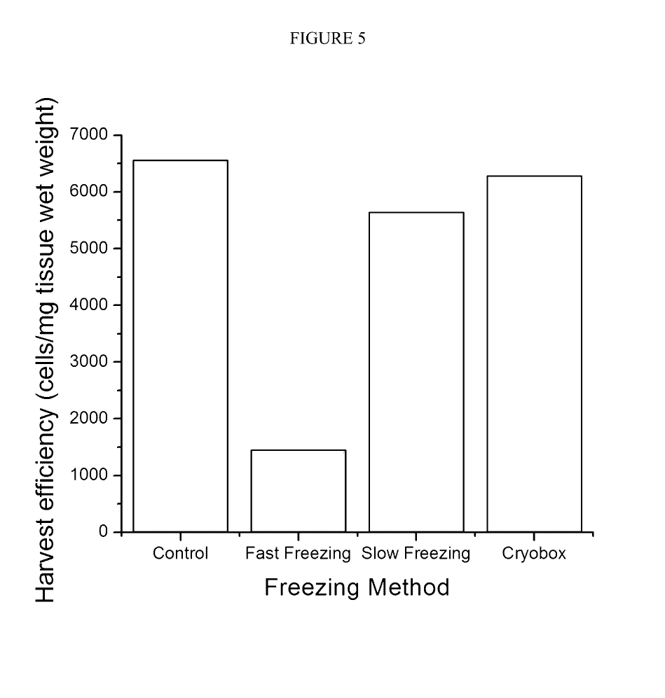

We found that cryopreserving tissue using the cryobox method was the most efficient method to obtain viable cells of all the methods (FIG. 5), but of course the techniques may vary or be further developed. When we compared our cryobox method to fresh tissue for cell morphology using fluorescent F-actin staining, we found that the cells were similar in terms of their morphologies and cytoskeletons (not shown). These results demonstrate that uterine tissue extracted in the delivery room could be cryopreserved with minimal negative effect on the myometrial smooth muscle cells.

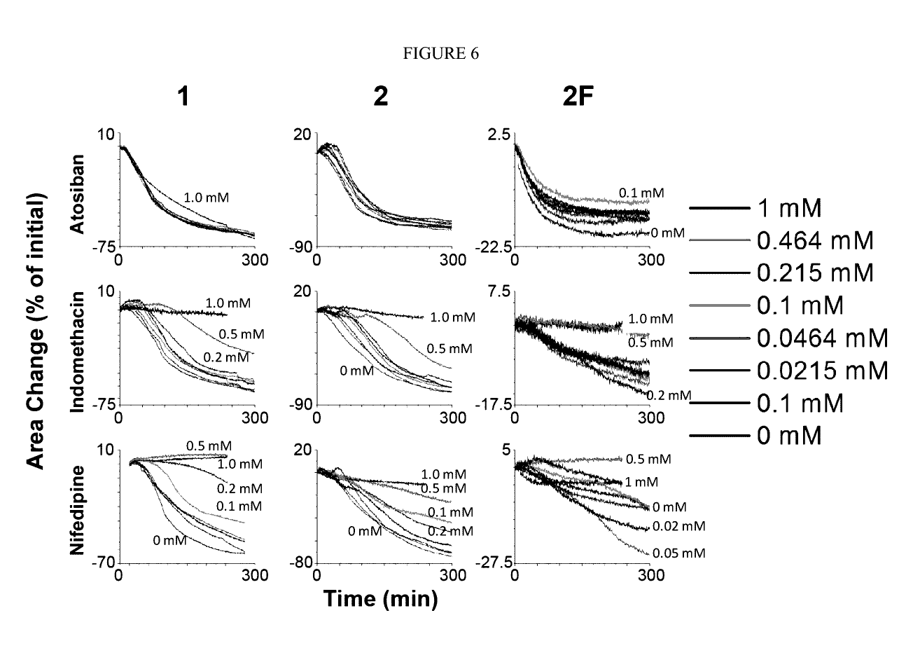

We also determined whether cryopreservation would have an effect on cell responsiveness in the contractile assay. We took cells from three different patients, and for two (patients 2 and 3), we cryopreserved their tissue using the cryobox method and harvested their cells (2F and 3F) after 3 weeks. All cell types were successfully printed into myometrial smooth muscle rings (not shown). When exposed to tocolytic drugs, ring contraction slowed, similarly to response found in our prototype experiments with commercially available SMCs (FIG. 6). Moreover, the cells from cryopreserved tissue and fresh tissue provided similar responses to agents. Thus, the freezing appeared to have no deleterious effect.

In using patient-derived smooth muscle cells, both fresh and cryopreserved, we demonstrated our ability to detect patient-specific responses, and that cryopreservation has minimal effect on both cell morphology, viability, and response to tocolytic drugs.

Ex vivo tissue was also tested for comparison to the CBiO Assay. Uterine biopsies obtained from cesarean sections were cut into myometrial strips (10×2×2 mm) mounted in an organ chamber system bath (10 mL) containing Kreb's buffer maintained at 37° C. and bubbled with 5% CO₂/95% O₂ to maintain the pH at 7.4. Eight uterine strips were obtained form each individual biopsy.

The strips were equilibrated for 1 h starting from a resting tension of 1 g and spontaneous uterine contractility was stabilized for isometric tension recording. Next, a dose response curve to indomethacin, nifedipine, atosiban, or saline (concentration 10⁻⁸ to 10⁻⁵ M) on spontaneous and oxytocin (10⁻⁸ M) induced contraction were performed. Two uterine strips were exposed to each tocolytic drug, thereby conducting duplicate experiments for each drug. To confirm tissue viability, potassium chloride (120 mM) was added to each chamber and contractility was recorded for 30 min (not shown). All tocolytic doses used were based from previous human myometrial contractility studies. As a result, we demonstrated our ability to detect tocolytic efficacy in ex vivo tissue (data not shown), which will be directly compared to the CBiO Assay in the next phase of our work.

The next study directly compared the ex vivo contractility of a patient-derived uterine tissue to the CBiO Assay conducted on the cells isolated from the biopsy taken from the same patient. For this purpose, uterine biopsies were obtained from two patients who had elective caesarean delivery and the following samples were isolated: (1) myometrial strips for uterine the myograph organ bath system (as described above); (2) myometrial cells for CBiO assay (as described above).

For the ex vivo tissue contractile activity, myometrial strips were mounted in a myograph organ bath system. The myometrial contraction of the strips was assessed in response to incubation with 1 μM-1 mM indomethacin (IND) or nifedipine (NIF), and the area under the curve (AUC) was analyzed.

From the same tissue, cells were grown in vitro as monolayer cultures and then harvested. The cells were magnetized with NanoShuttle, and printed into 3D rings using 75,000 cells per 384 well, cultured while levitated for 3 hours. After the drug was added, the magnet removed, and ring contraction was tracked for 5 h with the iPod Touch, as described above.

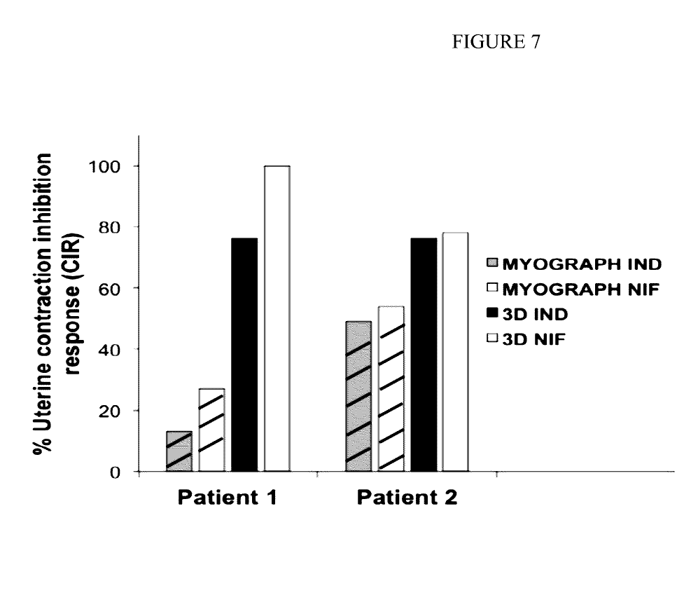

The contractile inhibitory response was the area change at 5 h for the CBiO Assay, and the AUC for myography (FIG. 7). The contractile inhibitory response (CIR) in this experiment was defined as the percent change in ring image pixels before and after drug exposure. Thus, FIG. 7 reports a change in size rather than a rate. However, rates can be reported if desired.

The preference for a tocolytic drug was determined by the IND/NIF preference ratio or “PR”. A PR<0.80 favors NIF; PR=0.81-1.20 no preference; PR >1.21 favors IND). Patient 1 displayed a PR of 0.48 with myography and 0.75 with CBiO Assay, while Patient 2 showed no preference in drug with a 0.92 with myography and a 0.97 with the CBiO Assay. These results show that the CBiO Assay was able to identify the drug of choice for the patient. As a result, we demonstrated our ability to detect tocolytic efficacy in the CBiO Assay that compares with ex vivo tissue.

Follow up will further confirm these results with clinical data, but this is not yet available. Our ability to collect clinical data assumes that the same patients will undergo at least one additional pregnancy, and have difficulty with preterm labor necessitating the use of the selected drug. Obviously, such data may take some years to collect, and work is expected to be ongoing for some time.

We have shown proof of concept for contractility assays, as well as proof of concept for use in endometrial muscle tissue from patients. Further, we have shown in a direct comparison, that the results track the results obtainable with ex vivo tissues from the same patient. Yet, our assay is simpler, faster, and easier to perform. More important it uses far less tissue—a scarce patient derived resource.

### Prophetic: Supply Chain for SMSS

In this study, we will focus on the supply chain to bring patients' myometrial smooth muscle cells to the lab. First, the isolation of myometrial smooth muscle cells will be optimized. Next, protocols for cryopreserving uterine tissue will be developed. Lastly, we will determine the effect of cyropreservation on cell phenotype. When the CBiO Assay is offered for service, these protocols will be used to bank and deliver cells to a service provider. The result of this study will be a clear process with the highest standards of sterility to bank, isolate, and deliver cells.

We will first determine the best methods to isolate uterine smooth muscle cells from biopsies, the primary source of cells for the CBiO Assay. Biopsy segments designated for the CBiO Assay will be washed in Hank's Balanced Salt Solution (HBSS), then digested stepwise with trypsin (0.05, 0.1, 0.2%), DNAse (0.05, 0.1, 0.2%), and collagenase type I (0.1, 0.2, 0.4%) for either 30, 60, and 120 min at 37° C. The cells will then be filtered, centrifuged, resuspended in smooth muscle cell medium (SMCM, PromoCell), then assessed for viability (CellTiter-Glo, Promega, WI). Based on the viability, the enzyme concentrations and digestion time will be chosen. As a negative control for later validation of the CBiO Assay, cells harvested from placental tissue will also be harvested, although these cells are vascular in nature and are thus not expected to match myometrial smooth muscle cells in response.

Cryopreserving uterine biopsies will be important to this assay so that these cells can be screened at later times when the mother expects another pregnancy. Tissues will be excised as above, and segmented into smaller 1 mm³ pieces. The key parameters for cryopreservation will be the choice of cryoprotecting agent and concentration. The cyroprotecting agents to be tested will be dimethyl sulfoxide (DMSO, 0.5 M, 1 M, and 2 M), polyethylene glycol (PEG, 20 kDa, 2.5%, 5%, and 10% w/v) and glycerol (20%, 40%, and 80% w/v). It should be noted that these concentrations are higher than what is normally used for cells, given that the cryoprotectants must penetrate through the dense tissue.⁵⁵ These cryoprotectants will either be dissolved in either SMCM, SMCM+10% fetal bovine serum (FBS), or FBS. After 7 days of cryopreservation, the tissues will be thawed and digested, then assayed for viability as was performed above. The cryoprotectant, concentration, and solvent will be selected based on cell viability.

After determining the ideal cryopreservation protocol that results in maximum viability, we will characterize the effect of cryopreservation on cell phenotype. Uterine biopsies will be cryopreserved and digested into myometrial smooth muscle cells as developed in above. These cells will then be plated in 2D in 96-well plates at a concentration of 5,000 cells/well using SMCM.

After 24 hours of adhesion, the cells will be fixed with 4% paraformaldehyde for 15 min for immunocytochemistry. Once fixed, the cells will be permeabilized with 0.2% Triton X-100, blocked with 1% donkey serum, then stained with the primary antibody solution (manufacturer's recommendations) overnight for 4° C. The next day, the primary antibodies will be fluorescently tagged with a secondary antibody (AlexaFluor, ThermoFisher) and nuclei will be counterstained by DAPI (KPL, MD), then imaged under fluorescence. The antigens to be tested are α-smooth muscle actin (α-SMA, Abcam, Cambridge, UK) and F-actin (ThermoFisher).

Expected Results:

We expect to successfully develop protocols to cryopreserve uterine tissues and isolate myometrial smooth muscle cells from them that have maintained their native phenotype. In harvesting myometrial smooth muscle cells, it should be noted that there is the potential contamination of these cells with uterine endothelial cells. While these cells will be sparse and eventually transdifferentiate into smooth muscle, their presence may still be a concern. An intermediate step to remove endothelial cells with 450 U/mL collagenase II could be added if needed.⁴² Trypsin is known to be a harsh enzyme, so other protocols from literature utilizing more specific enzymes, such as collagenase, could be used.⁵⁶

Another possible cell source is placental tissue, in which the cells are more vascular in nature, but could still be used to screen tocolytic therapies. For cryopreservation, while the focus is on maintaining cell viability, one protocol could be found to have high viability but alter phenotype. These two factors must be balanced: on one hand, the scarcity of the tissue demands a high viable cell yield, while at the same time the cells must maintain their phenotype. Thus, we expect to do experiments in parallel to find the best combination. The expected result of this study is a supply chain to harvest a patient's cells for screening in the CBiO Assay.

### Prophetic: Optimize CBiO Assay for 384-Well Formats

In this work, we will optimize the CBiO Assay to 384-well formats. We originally used a 384-well format to develop the CBiO Assay, but the assay as developed was limited by cell number, which required 1×10⁵ cells/ring for the iPod to detect the ring. In contrast, spheroids in 384-well plates can be formed with as few as 1,000 cells, suggesting that robust rings can also be made with fewer cells. The number of cells is important given that myometrial SMCs from specific patients are scarce resources. This study will first design a new magnetic drive to require far less cells. Next, we will assess the ability of these rings to detect tocolytic efficacy. As a result of this work, we will have a 384-well format that requires fewer cells and takes full advantage of scarce resources, such as uterine biopsy tissue.

A new magnetic drive will be made with smaller ring magnets (0.0625″ OD×0.03125″ ID) than the original magnets (0.125″ OD×0.0625″ ID). This drive will be rapid prototyped and assembled, and quality will be assessed empirically by the ability of the magnetic drive to attract cells magnetized with NS (Nano3D Biosciences) to the bottom of a 384-well cell-repellent plate (all plates are from Greiner Bio-One, Germany).

Once validated for quality, we will determine the optimal cell number to be detected by the iPod or other smart phone, tablet or pad. Uterine smooth muscle cells (PromoCell) will be magnetized with NS overnight. The next day, the cells will be detached, counted, resuspended in SMCM, and levitated in cell-repellent 6-well plates at 3.2×10⁶ cells/well for 2 h to build ECM. Afterwards the levitated samples will be disrupted and distributed into a cell-repellent 384-well plate at 5, 10, and 20×10³ cells/well.

The magnetized cells will then be printed into rings by placing the plate atop the new magnetic drive to form one ring per well. After 1 h, the plate will be removed and assessed for whether the cells formed competent rings. Next, the rings will be imaged with the iPod to determine whether the rings can be imaged with sufficient resolution using the n3Dock. The lowest concentration (or cell number) that meets these two criteria will be used.

Once optimized for cell concentration, the CBiO Assay will be performed with this new format to confirm that smaller rings can still detect tocolytic efficacy. Uterine smooth muscle cells (PromoCell) will be printed as was optimized. Before removing the plate off the magnet to allow rings to contract, tocolytic drugs will be added, either: atosiban, indomethacin, or nifedipine. Ibuprofen will be used as a positive control. Once added, the plate will be removed off the magnet and moved to the n3Dock in a humidified environment (37° C., 5% CO₂) for imaging every 4 min for 24 h. The next day, the images will be moved off the iPod onto a computer for analysis using custom image analysis software written in Python. The dose-dependent response will be fitted and analyzed for statistics (OriginPro, Origin Lab, Northampton, Mass.) to determine IC₅₀'s and statistical significance.

Expected Results:

We expect to successfully design a magnetic drive with a smaller ring and find the ideal cell/ring concentration to perform the CBiO Assay in 384-well plates. Our experience with 384-well formats has been that 10×10³ cells/well for spheroids is the lowest limit that can be imaged and resolved on the iPod. With a ring shape, we expect a larger cell number to yield a competent ring that can be detected. Otherwise, the ring is too sparse and transparent, and unable to be imaged, although the use of a better optical system might still allow the use of fewer cells. Thus, we believe 10-50×10³ cells/well or about 20-30×10³ cells/well or about 25×10³ or less will be the ideal cell concentration. Moreover, we expect these rings to detect tocolytic efficacy as was shown above with larger rings. We expect to yield a fully optimized CBiO Assay protocol for 384-well plates that uses far less cells than our prototype work.

### Prophetic: Compare CBiO with Ex Vivo Uterine Tissue

This study will compare the myometrial smooth muscle rings for the CBiO Assay with the original tissue. The two models will be compared by both immunohistochemistry and gene expression analysis. The goal is to demonstrate that 3D rings of myometrial smooth muscle rings printed with magnetic 3D bioprinting accurately represent the native uterine tissue.

Uterine biopsies will be obtained, frozen, and thawed. 25% of the biopsy will be digested for myometrial smooth muscle cells. Then, these cells will be printed into 3D rings using optimized the cell/ring concentration, and immediately fixed in 4% paraformaldehyde overnight. Concomitantly, another 25% of these biopsies will be immediately fixed in 4% paraformaldehyde overnight, paraffin embedded, and sectioned for immunohistochemistry.

Both 3D rings and biopsies will then be stained for the antigens: collagen type I, fibronectin, αSMA, oxytocin receptor, and prostaglandin-E2. These tissues will undergo antigen retrieval with a citrate buffer solution (Antigen Decloaker, Biocare Medical, CA). After antigen retrieval, the samples will be blocked, stained with the primary antibody according to manufacturer's directions, then the secondary antibody, and counterstained by DAPI for nuclei. The rings and biopsy slides will then be imaged under fluorescence.

Uterine biopsies will be processed into both myometrial smooth muscle rings and tissue. Both tissues will be dried and frozen at −80° C. to lyse the cells. The tissues will then be outsourced for RNA isolation and genome-wide expression analysis (LC Sciences, Houston, Tex.). Genes related to smooth muscle contraction will be filtered and analyzed, to view any differences in gene expression between the two tissues. Myometrial SMCs grown in 2D will serve as a control.

Expected Results:

We expect to see a general similarity between myometrial smooth muscle rings and the original tissue they were derived from in both phenotype and gene expression. If the myometrial smooth muscle rings do deviate, it is likely a result of the lack of ECM organization that is seen in the uterus. Other antigens to focus on include ECM-related antigens such as collagen (types I, III, IV, V, and VI) and fibronectin.⁵⁷ The expected result is that myometrial smooth muscle rings are close to the uterus in structure, phenotype, and gene expression.

### Prophetic: Compare CBiO, Ex Vivo Tissue, and Clinical Data

The goal herein is to compare the drug responses of in vitro myometrial smooth muscle rings made according to these methods with patient-derived ex vivo uterine tissue and with actual clinical data. In vitro myometrial smooth muscle rings will be assayed using the CBiO Assay in 384-well plates as optimized above. Uterine tissue strips will be used in the organ bath system to assess contractility. Lastly, these results will be compared to patient responses to tocolytic drugs based on clinical data. The goal hereunder is to confirm that the responses seen in the CBiO assay align with current methods of assessing uterine contractility both ex vivo and clinically. With luck, the CBiO will provide better results that the ex vivo uterine strip assay, but even if the results are comparable, the technical ease of the assay provides tremendous advantages.

Myometrial smooth muscle cells will be harvested, then printed into rings in 384-well plates as described above. After printing, drugs will be added at varying concentrations (Table 1). The plate will then be removed off the magnet and moved to an n3Dock in a humidified environment (37° C., 5% CO₂). The iPod Touch or other suitable instrument will be programmed to image the rings every 4 min for 24 h. The next day, the images will be moved from the iPod to a computer, where the change in ring size and the rate of contraction is measured and plotted using custom Python-based image analysis software. The dose-dependent response will be fitted and analyzed for statistics (OriginPro, Origin Lab, Northampton, Mass.) to determine IC₅₀'s and statistical significance.

TABLE 1
Drugs to be tested and the concentration ranges for the various assays.

| | | | Clinical Treatment of Preterm Labor | | | |
|---|---|---|---|---|---|---|
| | | | | Cmax | Onsef | Delay in |
| | CBiO Assay | Organ chamber | | (Peak plasma | of | delivery for |
| Drug | concentration | concentration | Dose | concentration) | action | 48 hrs |
| Indomethacin | 0.01 to 1 mM | 0.01 to 10 μM | 50 mg oral, then 25 | 2 μg/ml* | 2 hrs* | 90-94%⁵⁸⁻⁶¹ |
| | | | mg every 6 hrs | | | |
| Nifedipine | 0.01 to 1 mM | 0.01 to 10 μM | 10 mg oral every 6 | 27 ng/ml* | 30 min* | 70%⁶² |
| | | | hrs | | | |
| Atosiban | 0.01 to 1 mM | 0.01 to 10 μM | 6.75 mg IV, then | 442 ng/ml⁶³ | 1 hr⁶³ | 93%⁶⁴ |
| | | | 300 mcg/min | | | |

*as recommended by the manufacturer

Our established ex vivo human model of uterine contractility will be used to measure the contractile inhibition of tocolytic drugs according to Table 1. 25% of the uterine biopsies obtained from EXP. 1 will be cut into strips (10×2×2 mm), mounted in an organ chamber system bath (10 mL) containing Kreb's buffer maintained at 37° C. and bubbled with 5% CO₂/95% O₂ to maintain the pH at 7.4.

Eight uterine strips will be obtained from each individual biopsy and equilibrated for 1 h starting from a resting tension of 1 g to stabilize spontaneous uterine contractility for isometric tension recording. Next, a dose response curve to tocolytic agents on spontaneous and oxytocin-induced (0.01 μM) contraction will be performed. Two uterine strips will be exposed to each tocolytic drug thereby conducting duplicate experiments for each drug. To confirm tissue viability, potassium chloride (120 mM) will be added in each chamber and contractility will be recorded for 30 min. All doses used are based on previous human myometrial contractility studies.⁴⁹⁻⁵⁴ Oxytocin and indomethacin (Sigma-Aldrich, MO) will be initially dissolved in water and ethanol, respectively. The final concentration of ethanol in the bath (130 μM) will be 130 times less than a plasma concentration that could possibly account for a tocolytic effect.⁶⁵

Isometric tension will be measured with isometric force transducers (Harvard Apparatus, MA) connected to a computer for data acquisition and analysis (Windaq, Dataq Instruments, OH). The area under the curve (AUC) will be defined as uterine contraction integral activity (IA) over a 30 min period and will be used as a measure of contractility. Baseline activity will be defined as the integral activity over the 30 min following stabilization of uterine contractions. The inhibitory effect of each drug will be determined by calculating the integral activity normalized to baseline integral activity over 30 min after each agent, then normalized to temporal control. From this data, the inhibitory response and EC₅₀ will be generated (Sigma Plot and GraphPad Prism, GraphPad Software, CA).

Clinical data will be obtained from the patient medical chart, including which individual tocolytic drug was used. The contractile pattern obtained from the tocodynamometer 1 h before and after each tocolytic drug administration will be collected over a 24 h period of drug exposure. The number of contractions during each period will be measured. The ratio of contractions before and after each drug administration will be used to represent the percent change in uterine contractility or inhibitory effect, and therefore, response to the tocolytic drug.

The inhibitory effect of each tocolytic drug obtained from the CBiO Assay, organ tissue system, and clinical data will be compared using one-way ANOVA. Since the current tocolytic therapy used in the United States includes only indomethacin and nifedipine, the ratio of inhibitory effect of indomethacin to nifedipine will be calculated. A ratio >1.2 would then suggest that indomethacin response is greater than nifedipine, whereas a ratio of <0.8 would suggest that nifedipine response is greater than indomethacin. A ratio between 0.8-1.2 suggests both drug responses are similar. Other information including the inhibitory effects of each drug over time, maximal effect and minimal effect will also be collected and analyzed.

Expected Results:

Our expectation is that the three sets of data show similarity in response to the various tocolytic therapies. Deviation likely will occur in the degree of response between the three sets, which will determine whether the CBiO Assay would be used to determine which therapy is most effective or which therapy and dosage is most effective. In addition to inhibitory effect of the tocolytic drug, other parameters including the rate of contraction and total contraction of the rings would be analyzed to determine trends. We expect the results of the CBiO Assay to correlate closely with organ chamber and clinical data so that it can be used to predict both the preferred therapy and dosage for preterm labor management.

The following references are incorporated by reference in their entirety:
22. Bell, E., Ivarsson, B. & Merrill, C. Production of a tissue-like structure by contraction of collagen lattices by human fibroblasts of different proliferative potential in vitro. Proc. Natl. Acad. Sci. U.S.A 76, 1274-8 (1979).
23. Shi, Y. & Vesely, I. Fabrication of mitral valve chordae by directed collagen gel shrinkage. Tissue Eng. 9, 1233-42 (2003).
24. Nirmalanandhan, V. S., Duren, A., Hendricks, P., Vielhauer, G. & Sittampalam, G. S. Activity of anticancer agents in a three-dimensional cell culture model. Assay Drug Dev. Technol. 8, 581-90 (2010).
25. Cuchiara, M. P., Allen, A. C. B., Chen, T. M., Miller, J. S. & West, J. L. Multilayer microfluidic PEGDA hydrogels. Biomaterials 31, 5491-7 (2010).
26. Xu, X. & Prestwich, G. D. Inhibition of tumor growth and angiogenesis by a lysophosphatidic acid antagonist in an engineered three-dimensional lung cancer xenograft model. Cancer 116, 1739-50 (2010).
27. Del Duca, D., Werbowetski, T. & Del Maestro, R. F. Spheroid preparation from hanging drops: Characterization of a model of brain tumor invasion. J. Neurooncol. 67, 295-303 (2004).
28. Tung, Y.-C. et al. High-throughput 3D spheroid culture and drug testing using a 384 hanging drop array. Analyst 136, 473-8 (2011).
29. Ivanov, D. P. et al. Multiplexing spheroid volume, resazurin and acid phosphatase viability assays for high-throughput screening of tumour spheroids and stem cell neurospheres. PLoS One 9, (2014).
30. Yoshii, Y. et al. High-throughput screening with nanoimprinting 3D culture for efficient drug development by mimicking the tumor environment. Biomaterials 51, 278-89 (2015).
31. Montanez-Sauri, S. I., Beebe, D. J. & Sung, K. E. Microscale screening systems for 3D cellular microenvironments: platforms, advances, and challenges. Cell. Mol. Life Sci. 72, 237-49 (2015).
32. Souza, G. R. et al. Three-dimensional tissue culture based on magnetic cell levitation. Nat. Nanotechnol. 5, 291-6 (2010).
33. Molina, J. R., Hayashi, Y., Stephens, C. & Georgescu, M.-M. Invasive glioblastoma cells acquire stemness and increased Akt activation. Neoplasia 12, 453-63 (2010).
34. Xu, L., Gao, G., Ren, J., Su, F. & Zhang, W. Estrogen Receptor β of Host Promotes the Progression of Lung Cancer Brain Metastasis of an Orthotopic Mouse Model. J. Cancer Ther. 3, 352-8 (2012).
35. Lee, J. S., Morrisett, J. D. & Tung, C.-H. Detection of hydroxyapatite in calcified cardiovascular tissues. Atherosclerosis 224, 340-7 (2012).
36. Becker, J. L. & Souza, G. R. Using space-based investigations to inform cancer research on Earth. Nat. Rev. Cancer 13, 315-27 (2013).
37. Daquinag, A. C., Souza, G. R. & Kolonin, M. G. Adipose tissue engineering in three-dimensional levitation tissue culture system based on magnetic nanoparticles. Tissue Eng. Part C. Methods 19, 336-44 (2013).
38. Tseng, H. et al. Assembly of a three-dimensional multitype bronchiole coculture model using magnetic levitation. Tissue Eng. Part C. Methods 19, 665-75 (2013).
39. Haisler, W. L. et al. Three-dimensional cell culturing by magnetic levitation. Nat. Protoc. 8, 1940-9 (2013).
40. Timm, D. M. et al. A high-throughput three-dimensional cell migration assay for toxicity screening with mobile device-based macroscopic image analysis. Sci. Rep. 3, 3000 (2013).
41. Castro-Chavez, F., Vickers, K. C., Lee, J. S., Tung, C.-H. & Morrisett, J. D. Effect of lyso-phosphatidylcholine and Schnurri-3 on osteogenic transdifferentiation of vascular smooth muscle cells to calcifying vascular cells in 3D culture. Biochim. Biophys. Acta 1830, 3828-34 (2013).
42. Tseng, H. et al. A three-dimensional co-culture model of the aortic valve using magnetic levitation. Acta Biomater. 10, 173-82 (2014).
43. Jaganathan, H. et al. Three-Dimensional In Vitro Co-Culture Model of Breast Tumor using Magnetic Levitation. Sci. Rep. 4, 6468 (2014).
44. Tseng, H. et al. A spheroid toxicity assay using magnetic 3D bioprinting and real-time mobile device-based imaging. Sci. Rep. In press (2015).
45. Jorge, S., Chang, S., Barzilai, J. J., Leppert, P. & Segars, J. H. Mechanical Signaling in Reproductive Tissues: Mechanisms and Importance. Reprod. Sci. 1933719114542023-(2014). doi:10.1177/1933719114542023
46. Seiler, A. E. M. & Spielmann, H. The validated embryonic stem cell test to predict embryotoxicity in vitro. Nat. Protoc. 6, 961-78 (2011).
47. Friedrich, J., Seidel, C., Ebner, R. & Kunz-Schughart, L. A. Spheroid-based drug screen: considerations and practical approach. Nat. Protoc. 4, 309-24 (2009).
48. Fischer, R. S., Wu, Y., Kanchanawong, P., Shroff, H. & Waterman, C. M. Microscopy in 3D: a biologist's toolbox. Trends Cell Biol. 21, 682-91 (2011).
49. Chioss, G. et al. In vitro myometrial contractility profiles of different pharmacological agents used for induction of labor. Am. J. Perinatol. 29, 699-703 (2012).
50. Chiossi, G. et al. Effect of maternal body mass index on in vitro response to tocolytics in term myometrium. Am. J. Obstet. Gynecol. 203, (2010).
51. Chiossi, G. et al. Does sildenafil citrate affect myometrial contractile response to nifedipine in vitro? Am. J. Obstet. Gynecol. 203, (2010).
52. Chiossi, G. et al. The effects of prostaglandin E1 and prostaglandin E2 on in vitro myometrial contractility and uterine structure. Am. J. Perinatol. 29, 615-621 (2012).
53. Chiossi, G. et al. Does preincubation with prostaglandin E1 or prostaglandin E2 enhance oxytocin-induced myometrial contractility in vitro? Am. J. Perinatol. 30, 649-654 (2013).
54. Longo, M. et al. Effects of L-type Ca2+-channel blockade, K+ ATP-channel opening and nitric oxide on human uterine contractility in relation to gestational age and labour. Mol. Hum. Reprod. 9, 159-164 (2003).
55. Müller-Schweinitzer, E. Cryopreservation of vascular tissues. Organogenesis 5, 97-104 (2009).
56. Mosher, A. A. et al. Development and validation of primary human myometrial cell culture models to study pregnancy and labour. BMC Pregnancy Childbirth 13 Suppl 1, S7 (2013).
57. Leppert, P. C., Jayes, F. L. & Segars, J. H. The extracellular matrix contributes to mechanotransduction in uterine fibroids. Obstet. Gynecol. Int. 2014, 783289 (2014).
58. Morales, W. J. & Madhav, H. Efficacy and safety of indomethacin compared with magnesium sulfate in the management of preterm labor: a randomized study. Am. J. Obstet. Gynecol. 169, (1993).
59. Morales, W. J., Smith, S. G., Angel, J. L., O'Brien, W. F. & Knuppel, R. A. Efficacy and safety of indomethacin versus ritodrine in the management of preterm labor: a randomized study. Obstet. Gynecol. 74, (1989).
60. Besinger, R. E., Niebyl, J. R., Keyes, W. G. & Johnson, T. R. Randomized comparative trial of indomethacin and ritodrine for the long-term treatment of preterm labor. Am. J. Obstet. Gynecol. 164, (1991).
61. Bivins, H. A., Newman, R. B., Fyfe, D. A., Campbell, B. A. & Stramm, S. L. Randomized trial of oral indomethacin and terbutaline sulfate for the long-term suppression of preterm labor. Am. J. Obstet. Gynecol. 169, (1993).
62. King, J. F., Flenady, V. J., Papatsonis, D. N., Dekker, G. A. & Carbonne, B. Calcium channel blockers for inhibiting preterm labour. Cochrane Database Syst. Rev. CD002255 (2003). doi:10.1002/14651858.CD002255
63. Goodwin, T. M. et al. The pharmacokinetics of the oxytocin antagonist atosiban in pregnant women with preterm uterine contractions. Am. J. Obstet. Gynecol. 173, 913-917 (1995).
64. Treatment of preterm labor with the oxytocin antagonist atosiban: a double-blind, randomized, controlled comparison with salbutamol. Eur. J. Obstet. Gynecol. Reprod. Biol. 98, (2001).
65. Schrock, A., Fidi, C., Low, M. & Baumgarten, K. Low-dose ethanol for inhibition of preterm uterine activity. Am. J. Perinatol. 6, 191-195 (1989).
61/099,966, WO2010036957, U.S. Pat. No. 8,815,231, US20110286975, US20140322784, Systems and methods for magnetic guidance and patterning of cells and materials, Souza et al., Sep. 25, 2008.
61/245,846, WO2011038370A1, U.S. Pat. No. 8,883,471, US20120171744, US20150104844, Materials for magnetizing cells and magnetic manipulation, Souza, priority Sep. 25, 2009
61/372,164, WO2013019212, US2014220672, US20160137974, Souza, Hardware for Magnetic 3D Culture, Souza, priority Aug. 10, 2010.
61/438,310, WO2012106089, US2013280754, 3D CELL VIABILITY ASSAY, Souza, priority Feb. 1, 2011.
61/638,029, 61/637,108, WO2013163059, US2015091233, Phone Camera & Sample Stand, Souza, priority Apr. 23, 2012.
US2009137018, WO2005003332, Becker
US2005054101, WO2005010162, Felder
US2006063252, WO2004083412, WO2004083416, Ito

1) A contractility assay comprising:
a) obtaining contractile cells;
b) magnetizing said contractile cells;
c) magnetically creating a 3D ring of magnetized cells using a ring magnet, said 3D ring having a hole;
d) taking photomicrographs of said 3D ring of magnetized cells at least one or more times; and,
e) analyzing said photomicrographs to measure hole size or rate of change of hole size or both;
f) wherein hole size hole size or rate of change of hole size relates to contractility of said contractile cells.

2) The method of claim 1, further including preparing a plurality of 3D rings for use as control samples and a preparing a plurality of 3D rings for use as test samples, wherein a test agent is added to each of said test samples.

3) The method of claim 2, further including adding varying amounts of said test agent to said plurality of test samples.

4) The method of claim 3, comprising taking a plurality of photomicrographs of said plurality of test samples and said control samples at a plurality of times.

5) The method of claim 3, further comprising washing out said test agent and taking a further plurality of photomicrographs of said test samples and said control samples at a further plurality of times.

6) The method of claim 1, wherein cells are magnetized with a composition comprising: a) a negatively charged nanoparticle; b) a positively charged nanoparticle; and c) a support molecule, wherein one of said negatively charged nanoparticle or positively charged nanoparticle contains a magnetically responsive element, and wherein said support molecule holds said negatively charged nanoparticle and said positively charged nanoparticle in an intimate and disordered admixture, not a micelle.

7) The method of claim 6, wherein the support molecule comprises peptides, polysaccharides, nucleic acids, polymers, poly-lysine, fibronectin, collagen, laminin, BSA, hyaluronan, glycosaminoglycan, anionic, non-sulfated glycosaminoglycan, gelatin, nucleic acid, extracellular matrix protein mixtures, antibody, or mixtures or derivatives thereof, wherein said negatively charged nanoparticle is a gold nanoparticle, and wherein said positively charged nanoparticle is an iron oxide nanoparticle.

8) The method of claim 6, wherein the composition comprises poly-lysine, gold nanoparticles, and iron oxide nanoparticles.

9) The method of claim 1, wherein said contractile cells are obtained from a patient.

10) The method of claim 1, wherein said contractile cells are obtained from a maternal patient's uterus, myometrium, placenta, vasculature, or umbilical cord.

11) The method of claim 1, wherein said contractile cells are obtained from a patient's uterus.

12) A uterine contractility assay comprising:
a) obtaining smooth muscle cells (SMCs) from a uterus, myometrium, placenta, maternal vasculature, or umbilical cord;
b) magnetizing said SMCs to make magnetized cells;
c) magnetically creating a 3D ring of magnetized cells using a ring magnet, said 3D ring having a hole;
d) adding a test agent to said 3D ring of magnetized cells;
e) taking photographs of said 3D ring of magnetized cells at one or more times before and after adding said test agent; and,
f) analyzing said photographs to measure rate of change of hole size;
g) wherein rate of change of hole size relates to contractility of said SMCs in response to said test agent.

13) The method of claim 12, further including preparing a plurality of 3D rings for use as control samples and preparing a plurality of 3D rings for use as test samples, wherein a test agent is added to each of said test samples.

14) The method of claim 13, further including adding varying amounts of said test agent to said plurality of test samples.

15) The method of claim 14, comprising taking a plurality of photographs of said 3D rings at a plurality of times.

16) The method of claim 12, further comprising washing out said test agent and taking a further plurality of photographs of said 3D rings at a further plurality of times.

17) The method of claim 12, wherein said SMCs are magnetized with a composition comprising: a) a negatively charged nanoparticle; b) a positively charged nanoparticle; and c) a support molecule, wherein one of said negatively charged nanoparticle or positively charged nanoparticle contains a magnetically responsive element, and wherein said support molecule holds said negatively charged nanoparticle and said positively charged nanoparticle in an intimate admixture, not a micelle.

18) The method of claim 17, wherein the composition comprises poly-lysine, gold nanoparticles, and iron oxide nanoparticles.

19) The method of claim 11, wherein contraction is initiated by removing said ring magnet.

20) The method of claim 11, wherein contraction is initiated by adding a contractile agent or combination of agents.

21) An assay device comprising a microtiter plate having a plurality of wells, each well containing a culture medium containing a 3D ring of magnetic contractile cells.

22) The device of claim 21, wherein each said 3D ring of magnetic contractile cells is floating in said culture medium.

23) The device of claim 21, wherein said magnetic contractile cells are myometrial smooth muscle cells.

24) A uterine contractility assay comprising:
a) obtaining contractile cells from a myometrium of a patient's uterus, and optionally freezing said cells before step b);
b) allocating said contractile cells to a plurality of wells in a microtiter plate;
c) magnetizing said contractile cells;
d) magnetically creating a 3D ring of magnetized cells using a ring magnet, said 3D ring having a hole, said hole having a size;
e) adding a test agent to said 3D ring of magnetized cells;
f) initiating contraction by removing said ring magnet; either before or after step e);
g) taking photographs of an entirety of said microtiter plate at one or more times before and after adding said test agent; and,
h) analyzing said photographs to measure a rate of hole size contraction;
i) wherein a reduced rate of hole size contraction means said test agent inhibits contraction.

25) The method of claim 24, wherein said microtiter plate is a 384-well microtiter plate, and wherein 10-100×10³ cells/well are used to form said 3D ring.

26) The method of claim 24, wherein said cells are grown in 3D culture for 1-7 days to form a 3D spheroid and wherein said spheroid is broken up to provide contractile cells for said allocating step b.

27) The method of claim 24, wherein said 3D rings are cultured for 1-3 days before said adding step e.
